(19) United States
(12) Patent Application Publication
(10) Pub. No.: US 2025/0342910 A1
(43) Pub. Date: Nov. 6, 2025

(54) SAMPLE MATCHING USING SELECT SINGLE NUCLEOTIDE POLYMORPHISMS WITHIN TARGET REGIONS

(71) Applicant: MyOme, Inc., Menlo Park, CA (US)

(72) Inventors: Akash Kumar, Palo Alto, CA (US); Stephanie Greer, Palo Alto, CA (US)

(21) Appl. No.: 19/209,683

(22) Filed: May 15, 2025

Related U.S. Application Data
(63) Continuation of application No. PCT/US23/79775, filed on Nov. 15, 2023.
(60) Provisional application No. 63/384,046, filed on Nov. 16, 2022.

(51) Int. Cl. G16B 30/10; G16B 20/20

(52) U.S. Cl. CPC G16B 30/10; G16B 20/20

(57) ABSTRACT

Disclosed herein are methods of determining sample matches. An example method includes receiving short-read sequence data, wherein the short-read sequence data is generated from a first sample using a short-read sequencing technique. The method further includes identifying a plurality of short-read single nucleotide polymorphisms (SNPs) from the short-read sequence data and selecting one or more SNPs from the plurality of short-read SNPs. The method further includes receiving long-read sequence data, wherein the long-read sequence data is generated from a second sample using a long-read sequencing technique, wherein the long-read sequence data comprises sequence data for at least the one or more SNPs. The method further includes determining whether the first sample and the second sample match based on the short-read sequence data and the long-read sequence data.

### CROSS-REFERENCE TO RELATED APPLICATION

This application is a continuation of International Application No. PCT/US2023/79775, filed Nov. 15, 2023, which claims the benefit of U.S. Provisional Application No. 63/384,046, filed on Nov. 16, 2022, both of which are incorporated herein by reference in their entirety.

### TECHNOLOGICAL FIELD

The present disclosure relates in general to sample matching, and more specifically, to methods for sample matching using select single nucleotide polymorphisms (SNPs) within target regions.

### BACKGROUND

Whole genome sequencing (WGS) currently relies on well-established short-read sequencing technology to identify variants that are relevant for disease. Currently, short-read sequencing is used within clinical settings. Nanopore long-read sequencing has recently emerged as a newer technology that has primarily been used in the research community but has potential for clinical and other testing applications.

### BRIEF SUMMARY

As described above, short-read sequencing is currently used in clinical settings to identify variants that are relevant for disease. Typically, short-read sequencing uses fragmented and amplified portions of a sample to generate short order sequence data, such as on the order of 200 to 600 base pairs per sequencing. Nanopore long-read sequencing may serve as a supplemental and/or alternative method of sample sequencing that is capable of generating long order sequence data, such as on the order of 10 thousand base pairs or more.

Nanopore long-read sequencing has been explored primarily in research settings but has shown promise for clinical and other testing applications. Unlike traditional short-read sequencing, nanopore long-read sequencing can directly sequence native DNA strands without need of amplification, making it an attractive method for structural variant breakpoint detection. Additionally, short-read sequencing relies on sequence overlap between DNA fragments to reconstruct a whole genome and thus may be prone to miss important target regions and/or fail to detect desirable SNPs if this sequence overlap is misaligned.

As such, it is likely that nanopore long-read sequencing will gain popularity in testing applications, initially as a confirmatory application for variants identified by short-read WGS and later as part of a comprehensive suite of testing protocols. As with any multi-platform assay, it will therefore be essential to ensure no sample mix-ups have occurred between assays such that the correct sample data is being combined and compared. One method of confirming sample identities is by using select SNPs to uniquely identify samples from one another.

Embodiments described herein advantageously allow for the matching of samples sequenced using disparate techniques (e.g., short-read sequencing and long-read sequencing) and thus provide solutions to the above-described problems. In particular, short-read sequence data pertaining to a first sample and captured using a short-read sequencing technique may be received and used to identify SNPs of the sample. Based at least in part on these SNPs, and in some embodiments, various allele frequency data, one or more target regions may be determined for long-read sequencing of a second sample. Long-read sequence data pertaining to a second sample and captured using a long-read sequencing technique may then be received. The short-read sequence data pertaining to the first sample and the long-read sequence data pertaining to the second sample of the second sample may be compared to determine whether the first sample and the second sample match. As such, this technique allows for a determination to be made as to whether the two (or more) samples match and therefore maintains the integrity of sample and subsequent testing of the samples. Additionally, this technique leverages data generated by an existing pipeline (e.g., data generated from short-read sequencing and/or long-read sequencing) to assess whether sample swaps have occurred without additional wet or dry lab protocol.

As such, methods are provided herein for determining sample matches. The method includes receiving short-read sequence data, wherein the short-read sequence data is generated from a first sample using a short-read sequencing technique. The method further includes identifying a plurality of short-read SNPs from the short-read sequence data and selecting one or more SNPs from the plurality of short-read SNPs. The method further includes receiving long-read sequence data, wherein the long-read sequence data is generated from a second sample using a long-read sequencing technique, wherein the long-read sequence data comprises sequence data for at least the one or more SNPs. The method further includes determining whether the first sample and the second sample match based on the short-read sequence data and the long-read sequence data.

In addition, another method for determining sample matches is disclosed herein. The method includes performing a short-read sequencing technique on a first sample to generate short-read sequence data and identifying a plurality of short-read SNPs from the short-read sequence data. The method annotates each short-read SNP of the plurality of short-read SNPs with a corresponding allele frequency and selects one or more SNPs from the plurality of short-read SNPs. The method further includes determining one or more target regions for long-read sequencing based on the one or more SNPs.

Similarly, an apparatus for determining sample matches is disclosed herein. The example apparatus comprises a processor and a memory storing software instructions that, when executed by the processor, cause the apparatus to receive short-read sequence data, wherein the short-read sequence data is generated from a first sample using a short-read sequencing technique. The processor and the memory storing software instructions that, when executed by the processor, further cause the apparatus to identify a plurality of short-read SNPs from the short-read sequence data and select one or more SNPs from the plurality of short-read SNPs. The processor and the memory storing software instructions that, when executed by the processor, further cause the apparatus to receive long-read sequence data, wherein the long-read sequence data is generated from a second sample using a long-read sequencing technique, wherein the long-read sequence data comprises sequence data for at least the one or more SNPs. The processor and the memory storing software instructions that, when executed by the processor, further cause the apparatus to determine whether the first sample and the second sample match based on the short-read sequence data and the long-read sequence data.

In addition, a computer program product for determining sample matches is disclosed herein. The computer program product includes at least one non-transitory computer-readable storage medium storing software instructions that, when executed by an apparatus, cause the apparatus to receive short-read sequence data, wherein the short-read sequence data is generated from a first sample using a short-read sequencing technique. The software instructions, when executed by an apparatus, further cause the apparatus to identify a plurality of short-read SNPs from the short-read sequence data and select one or more SNPs from the plurality of short-read SNPs. The software instructions, when executed by an apparatus, further cause the apparatus to receive long-read sequence data, wherein the long-read sequence data is generated from a second sample using a long-read sequencing technique, wherein the long-read sequence data comprises sequence data for at least the one or more SNPs. The software instructions, when executed by an apparatus, further cause the apparatus to determine whether the first sample and the second sample match based on the short-read sequence data and the long-read sequence data.

The foregoing brief summary is provided merely for purposes of summarizing some example embodiments described herein. Because the above-described embodiments are merely examples, they should not be construed to narrow the scope of this disclosure in any way. It will be appreciated that the scope of the present disclosure encompasses many potential embodiments in addition to those summarized above, some of which will be described in further detail below.

### BRIEF DESCRIPTION OF DRAWINGS

Having described certain example embodiments in general terms above, reference will now be made to the accompanying drawings, which are not necessarily drawn to scale. Some embodiments may include fewer or more components than those shown in the figures.

FIG. 1 illustrates an example process overview for determining sample matches, which may be used in accordance with some example embodiments described herein.

FIGS. 2A-2D depict operational examples of the results of sample matching, in the form of heatmaps, as determined using techniques described herein.

FIG. 3 illustrates a schematic block diagram of an example device that may perform various operations in accordance with some example embodiments described herein.

FIG. 4 is a data flow diagram of some embodiments of a process for performing sample matching.

### DETAILED DESCRIPTION

Some example embodiments will now be described more fully hereinafter with reference to the accompanying figures, in which some, but not necessarily all, embodiments are shown. Because inventions described herein may be embodied in many different forms, the invention should not be limited solely to the embodiments set forth herein; rather, these embodiments are provided so that this disclosure will satisfy applicable legal requirements.

Many modifications and other embodiments of the disclosure set forth herein will come to mind to one skilled in the art to which this disclosure pertains having the benefit of the teachings presented in the foregoing description and the associated drawings. Therefore, it is to be understood that the embodiments are not to be limited to the specific embodiments disclosed and that modifications and other embodiments are intended to be included within the scope of the appended claims. Moreover, although the foregoing descriptions and the associated drawings describe example embodiments in the context of certain example combinations of elements and/or functions, it should be appreciated that different combinations of elements and/or functions may be provided by alternative embodiments without departing from the scope of the appended claims. In this regard, for example, different combinations of elements and/or functions than those explicitly describe herein are also contemplated as may be set forth in some of the appended claims. Although specific terms are employed herein, they are used in a generic and descriptive sense only and not for purposes of limitation.

### Definition of Certain Terms

Technical and scientific terms used herein have the meanings commonly understood by one of ordinary skill in the art to which the present invention pertains, unless otherwise defined. Materials to which reference is made in the following description and examples are obtainable from commercial sources, unless otherwise noted.

The terms “computer-readable medium” and “memory” refer to non-transitory storage hardware, non-transitory storage device or non-transitory computer system memory that may store computer-executable instructions or software programs that may be accessed by a controller, a microcontroller, a computational system or a module of a computational system. A non-transitory computer-readable medium may be accessed by a computational system or a module of a computational system to retrieve and/or execute the computer-executable instructions or software programs stored on the medium. Exemplary non-transitory computer-readable media may include, but are not limited to, one or more types of hardware memory, non-transitory tangible media (for example, one or more magnetic storage disks, one or more optical disks, one or more USB flash drives), computer system memory or random access memory (such as, DRAM, SRAM, EDO RAM), and the like.

The term “computing device” may refer to any computer embodied in hardware, software, firmware, and/or any combination thereof. Non-limiting examples of computing devices include a personal computer, a server, a laptop, a mobile device, a smartphone, a fixed terminal, a personal digital assistant (“PDA”), a kiosk, a custom-hardware device, a wearable device, a smart home device, an Internet-of-Things (“IoT”) enabled device, and a network-linked computing device.

The term “target region” refers to a genomic region that is targeted for enrichment. In some embodiments, a target region may refer to a genomic region that is targeted for enrichment by adaptive sampling with long-read sequencing, such as nanopore sequencing.

### Example Implementations

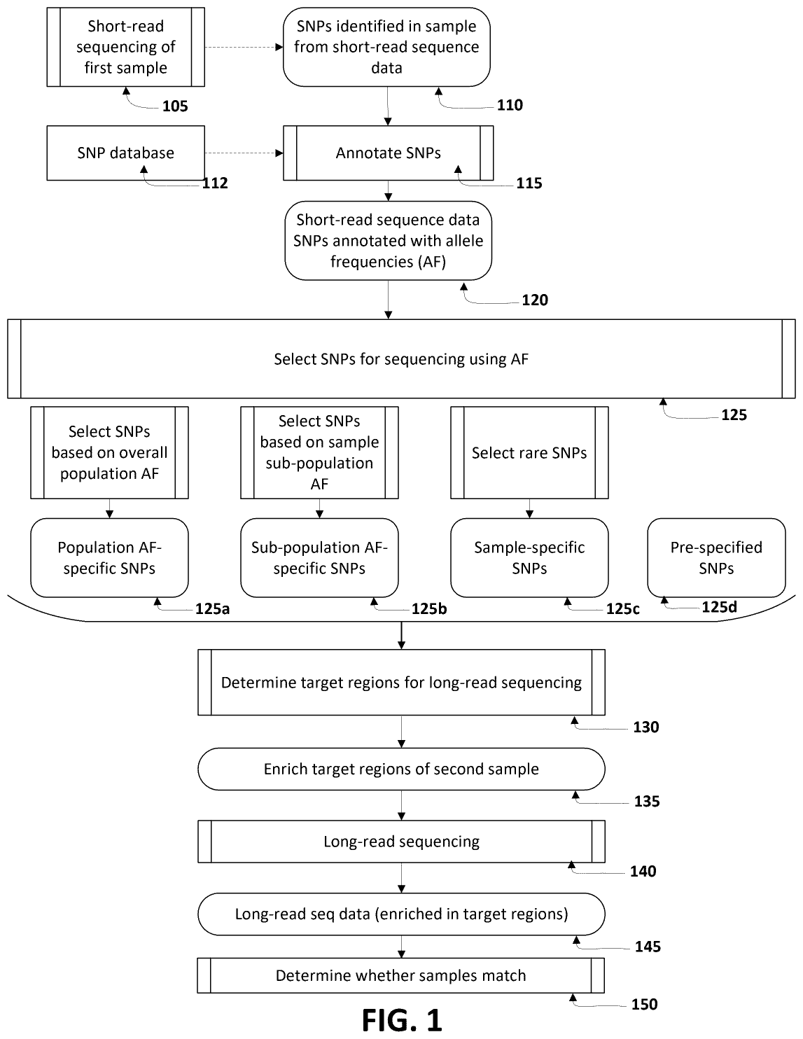

FIG. 1 illustrates an example process for matching samples. At operation 105, short-read sequencing data is obtained for a first sample by performing short-read sequencing on the first sample. In some embodiments, short-read sequencing includes (i) fragmenting the first sample into two or more fragments, (ii) amplifying the two or more fragments, and (iii) generating the short-read sequence data based on sequencing the two or more amplified fragments. In some embodiments, short-read sequencing includes performing Illumina sequencing (e.g., a short-read sequencing technique) on the first sample. The first sample may be a sample of a first subject and may be collected by any suitable method. In some embodiments, the first sample is DNA from a sample type (e.g., blood, tissue, cell line, etc.).

At operation 110, one or more short-read sequence SNPs are identified from the short-read sequence data. Each identified SNP is also associated with a SNP locus, which defines the genomic location of the SNP on the genome. A SNP may be a single base-pair change in a subject genome. As described above, one or more SNPs may be used to distinguish samples which are derived from unique individuals.

At operation 115, each of the one or more short-read sequence SNPs are annotated with an allele frequency. In some embodiments, an SNP database 112, such as Genome Aggregation Database (gnomAD), may be used to provide allele frequency (AF) for each SNP loci identified in operation 110. The SNP database may include AF values across an entire population and/or one or more sub-populations.

At operation 120, the one or more SNPs in the short-read sequence data SNPs are annotated aggregated to yield a complete short-read sequence data with each of the one or more SNPs annotated with AF.

At operation 125, one or more SNPs are selected for sequencing. The one or more SNPs may be selected based on one or more AF considerations. In some embodiments, a particular number of SNPs are selected. Furthermore, in some embodiments, a certain number of SNPs are selected per target region. For example, one SNP may be selected per target region such that the one selected SNP in each target region will be sequenced and compared to a corresponding sequenced SNP at the same SNP locus using a long-read sequencing technique. As another example, one hundred fifty SNPs may be selected such that each such that each of the one hundred fifty selected SNP in each target region will be sequenced and compared to a corresponding sequenced SNP at corresponding SNP loci using a long-read sequencing technique.

In particular, in some embodiments, the one or more SNPs which are selected are selected based on an overall population AF as shown in operation 125a. For example, the one or more SNPs may be selected such that there is no one majority of the population has a particular form the of the SNP. As another example, the one or more SNPs may be selected such that a minority of the population has a particular form the of the SNP. As such, one or more SNPs selected based on a population AF may be population AF-specific SNPs.

In some embodiments, the one or more SNPs which are selected are selected based on a sub-population AF as shown in operation 125b. For example, the one or more SNPs may be selected such that a majority of a particular sub-population has a particular form the of the SNP. As such, one or more SNPs selected based on a sub-population AF may be sub-population AF-specific SNPs.

In some embodiments, the one or more SNPs which are selected are selected based on a rare AF as shown in operation 125c. For example, the one or more SNPs may be selected such only a threshold percentage of a population or a particular sub-population has a particular form the of the SNP. As such, one or more SNPs selected based on a rare AF may be sample-specific AF-specific SNPs.

In some embodiments, the one or more SNPs which are selected are selected based on one or more pre-specified SNPs in operation 125d. For example, the one or more SNPs may be selected to include one or more pre-specific SNPs, which may be chosen by administrators, clinicians, geneticists, etc. As such, one or more SNPs selected according to chosen SNPs are pre-specified SNPs.

In some embodiments, the one or more SNPs are selected such that the distance between SNP loci is maximized. In some embodiments, a set number of SNPs may be selected and may thus control the number of SNPs which are selected based on an associated AF. The distance between candidate SNPs, which may be short-read SNPs which are selectable based on an associated AF, may be determined and optimized such that the distance between respective selected SNP loci is maximized. For example, the set number of SNPs may be 100 such that only 100 SNPs may be selected. However, 200 candidate SNPs may be identified based on annotating the short-read SNPs with their corresponding AF. A distance between combinations of the 200 candidate SNPs may then be determined using different selections of the 200 candidate SNPs for the selected SNPs until a distance between SNP loci is maximized.

At operation 130, one or more target regions are determined for long-read sequencing. The one or more target regions are determined based on the one or more selected SNPs. The one or more target regions may be determined such that each region includes a particular number of SNPs, as mentioned above. In some embodiments, each target region may uniquely identify a particular genomic region of a sample which is targeted for enrichment by adaptive sampling with nanopore sequencing. In some other embodiments, long-read whole genome sequencing instead of a targeted sequencing approach. In some embodiments, the one or more target regions are provided as a BED file for use in long-read sequencing, such as in targeted nanopore sequencing. As such, during operations involving the long-read sequencing of a sample, the one or more target regions may be used to determine which samples (e.g., strands of DNA and/or RNA) should be sequenced by an associated sequencer. The associated sequencer may use the one or more target regions of interest to identify which samples to sequence in real-time or near real-time and automatically eject the samples from the nanopore that do not include the target regions of interest or fully process the sample which includes the target regions of interest.

At operation 135, the one or more target regions of a second sample are enriched. In some embodiments, the nanopore signal is not informatically enriched at polymorphic sites in a target sample (e.g., the second sample).

At operation 140, long-read sequencing is performed on the second sample. In some embodiments, long-read sequencing includes performing nanopore sequencing on the second sample. The second sample may be a sample of a first or second subject and may be collected by any suitable method. In some embodiments, the second sample is DNA from a sample type (e.g., blood, tissue, cell line, etc.).

At operation 145, long-read sequence data for the second sample is determined based on the long-read sequencing of the second sample.

At operation 150, it is determined whether the first sample and the second sample are matching. The first sample and second sample may be determined to match based on the short-read sequence data pertaining to the first sample and the long-read sequence data pertaining to the second sample.

Once a determination has been made regarding whether the first sample and second sample match, a sample determination response may be generated and provided to one or more computing devices. The sample determination response may describe the determination of whether first sample and second sample match such that one or more end users who may receive the sample determination response, are informed of whether the first sample and second sample match.

In some embodiments, the number of SNP-matches between the first sample and second sample are determined. An SNP-match may be determined in an instance the SNP at a particular SNP locus as described by the short-read sequence data and the long-read sequence data are of the same SNP type. For example, an SNP-match is determined in an instance a short-read sequence data SNP value at a particular SNP locus is “A” and a long-read sequence data SNP value at the same SNP locus is also “A”.

In some embodiments, in order to determine whether the first sample and second sample match, the number of total SNP-matches must satisfy one or more SNP-match thresholds. For example, if 100 SNPs are selected for each sample (e.g., 100 SNPs values at fixed SNP loci are determined for the first sample and second sample), each corresponding SNPs between the short-read sequence data and long-read sequence data may be compared to determine whether an SNP-match exists for the SNP locus. In some embodiments, a SNP-match threshold may be 0.85 (e.g., 85%). As such, if 85 or more of the selected SNPs are SNP-matches, then the samples are determined to match.

In some embodiments, the one or more target regions described by the short-read sequence data and the long-read sequence data are compared to determine a number of target region-matches. A target region may be determined to match in an instance a threshold number or proportion of SNPs in the target region match. For example, a target region may include 100 SNPs. Each of the 100 SNPs of the target region are compared between the short-read sequence data of the first sample and the long-read sequence data of the second sample to determine whether the corresponding SNPs match. A number of target region SNP matches may be determined for the target region by summing the total number of SNP-matches in the target region. A target region-match may then be determined for the target region in an instance the number of target region SNP-matches satisfies one or more target region SNP-match thresholds. For example, a target region SNP-match threshold may be 0.85 (e.g., 85%), such that if 85 of 100 SNPs of a target region are determined to be SNP-matches, a target region-match is determined. In some other embodiments, other target region-match thresholds can be used. For example, if the proportion target regions that match (proportion of sites with matching genome types) have over 30× coverage, then a higher percentage match (e.g., 97-99%) can be used.

In some embodiments, in order to determine whether the first sample and second sample match, the number of target region-matches must satisfy one or more target region-match thresholds. For example, if 10 target regions are selected for each sample, the corresponding SNPs of the corresponding target regions may be compared to determine whether a target region-match exists. In some embodiments, a target region-match threshold may be 0.85 (e.g., 85%). As such, if 9 or more of the target regions are target region-matches, then the samples are determined to match.

### Example Implementation System

The methods described herein may be implemented on a variety of systems. For instance, in some embodiments the system may be used for receiving short-read sequence data pertaining to a first sample, identifying a plurality of short-read SNPs, selecting one or more SNPS, determining one or more target regions for long-read sequencing, receiving long-read sequence data pertaining to a second sample, determining whether the first sample and the second sample are matching, etc.

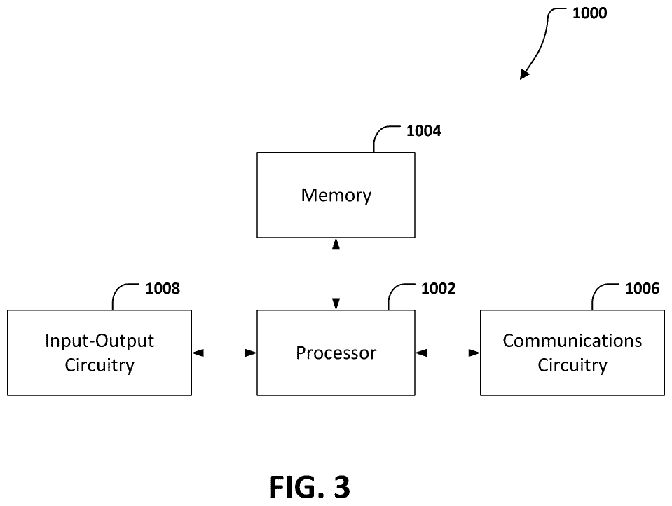

The system may include one or more system devices, which may be embodied by one or more computing devices or servers, shown as apparatus 1000 in FIG. 3. As illustrated in FIG. 3, the apparatus 1000 may include a processor 1002, memory 1004, and communications hardware 1006, each of which will be described in greater detail below. While the various components are only illustrated in FIG. 3 as being connected with apparatus 1000, it will be understood that the apparatus 1000 may further comprise a bus (not expressly shown in FIG. 3) for passing information amongst any combination of the various components of the apparatus 1000. The apparatus 1000 may be configured to execute various operations described above.

The processor 1002 (and/or co-processor or any other processor assisting or otherwise associated with the processor) may be in communication with the memory 1004 via a bus for passing information amongst components of the apparatus. The processor 1002 may be embodied in a number of different ways and may, for example, include one or more processing devices configured to perform independently. Furthermore, the processor may include one or more processors configured in tandem via a bus to enable independent execution of software instructions, pipelining, and/or multithreading. The use of the term “processor” may be understood to include a single core processor, a multi-core processor, multiple processors of the apparatus 1000, remote or “cloud” processors, or any combination thereof.

The processor 1002 may be configured to execute software instructions stored in the memory 1004 or otherwise accessible to the processor (e.g., software instructions stored on a separate storage device). In some cases, the processor may be configured to execute hard-coded functionality. As such, whether configured by hardware or software methods, or by a combination of hardware with software, the processor 1002 represents an entity (e.g., physically embodied in circuitry) capable of performing operations according to various embodiments of the present invention while configured accordingly. Alternatively, as another example, when the processor 1002 is embodied as an executor of software instructions, the software instructions may specifically configure the processor 1002 to perform the algorithms and/or operations described herein when the software instructions are executed.

The memory 1004 is non-transitory and may include, for example, one or more volatile and/or non-volatile memories. In other words, for example, the memory 1004 may be an electronic storage device (e.g., a computer readable storage medium). The memory 1004 may be configured to store information, data, content, applications, software instructions, or the like, for enabling the apparatus to carry out various functions in accordance with example embodiments contemplated herein.

The communications hardware 1006 may be any means such as a device or circuitry embodied in either hardware or a combination of hardware and software that is configured to receive and/or transmit data from/to a network and/or any other device, circuitry, or module in communication with the apparatus 1000. In this regard, the communications hardware 1006 may include, for example, a network interface for enabling communications with a wired or wireless communication network. For example, the communications hardware 1006 may include one or more network interface cards, antennas, buses, switches, routers, modems, and supporting hardware and/or software, or any other device suitable for enabling communications via a network. Furthermore, the communications hardware 1006 may include the processing circuitry for causing transmission of such signals to a network or for handling receipt of signals received from a network.

The communications hardware 1006 may be configured to provide output to a user and, in some embodiments, to receive an indication of user input. The communications hardware 1006 comprise a user interface, such as a display, and may further comprise the components that govern use of the user interface, such as a web browser, mobile application, dedicated user device, or the like. In some embodiments, the communications hardware 1006 may include a keyboard, a mouse, a touch screen, touch areas, soft keys, a microphone, a speaker, and/or other input/output mechanisms. The communications hardware 1006 may utilize the processor 1002 to control one or more functions of one or more of these user interface elements through software instructions (e.g., application software and/or system software, such as firmware) stored on a memory (e.g., memory 1004) accessible to the processor 1002.

### EXAMPLES

### Example 1

To showcase the accuracy and reliability of the above-described methods for matching samples, this method was applied to for five samples, each sequenced using short-read sequencing and long-read sequencing. In particular, five Coriell cell lines were sequenced using Illumina sequencing (e.g., short-read sequencing) and nanopore sequencing (e.g., long-read sequencing) to generate short-read sequence data and long-read sequence data, respectively.

First, Illumina sequencing was performed for each cell line to generate the short-read sequence data for each sample. Four SNP loci and by extension, four target regions, were selected based on the short-read sequence data. Prior to long-read sequencing, the four genomic regions corresponding to the four target regions of each sample were enriched and subsequently sequenced using nanopore sequencing to capture the long-read sequence data for each sample.

Table 1 shows the selected SNPs as captured by the short-read sequence data and the long-read sequence data for the NA09216 sample. As shown in Table 1, the genotypes between the long-read sequence data and the short-read sequence data were determined to match at each of the four SNP loci, thereby yielding a proportion of matching genotypes of 1. Therefore, there is a high probability the sample as sequenced by the short-read sequencing and long-read sequencing techniques were derived from the same individual and thus, the samples are matching.

TABLE 1

| | 10_{—} | 17_{—} | 8_{—} | 9_{—} |
|---|---|---|---|---|
| | 895999576_{—} | 41199913_{—} | 61701057_{—} | 137604936_{—} |
| | A_G | T_C | C_T | T_G |
| NA09216 | A/G | T/C | C/T | T/G |
| genotype | | | | |
| (short- | | | | |
| reads) | | | | |
| NA09216 | A/G | T/C | C/T | T/G |
| genotype | | | | |
| (long-reads) | | | | |
| Match? | Yes | Yes | Yes | Yes |
| Proportion | 1 (4/4) | | | |
| of matching | 100% | | | |
| genotypes | | | | |
| between | | | | |
| samples | | | | |

Additionally, Table 2 illustrates the proportion of target regions that match among the four target regions between the long-read sequence data and the short-read sequence data for the NA09216 sample. Here, 100 SNPs are considered in each 500 kilo base pair (kbp) target region but only SNP loci covered by at least 10 reads in both the short-read sequence and long-read sequence are selected for further processing. Within each target region, a threshold of the proportion of matching genotypes was taken to be 0.85 (e.g., 85%), such that this target region had 85% or higher of matching genotypes, the target region was taken to be matching. As shown in Table 2, again each of the four target regions were determined to be matching such that the proportion of matching genotypes was 1. Therefore, there is a high probability the sample as sequenced by the short-read sequencing and long-read sequencing techniques were derived from the same individual and thus, the samples are matching.

TABLE 2

| | | | | | | Number | Proportion |
|---|---|---|---|---|---|---|---|
| | | chr9_{—} | chr10_{—} | chr8_{—} | chr1_{—} | matching | Matching |
| Id1 (long-read seq) | Id2 (short-read seq) | region | region | region | region | regions | regions |
| NA09216_LONG | NA09216_SHORT | 0.96 | 1 | 0.97 | 0.97 | 4 | 1 (4/4) |
| | | (27/28) | (43/43) | (35/36) | (30/31) | | 100% |

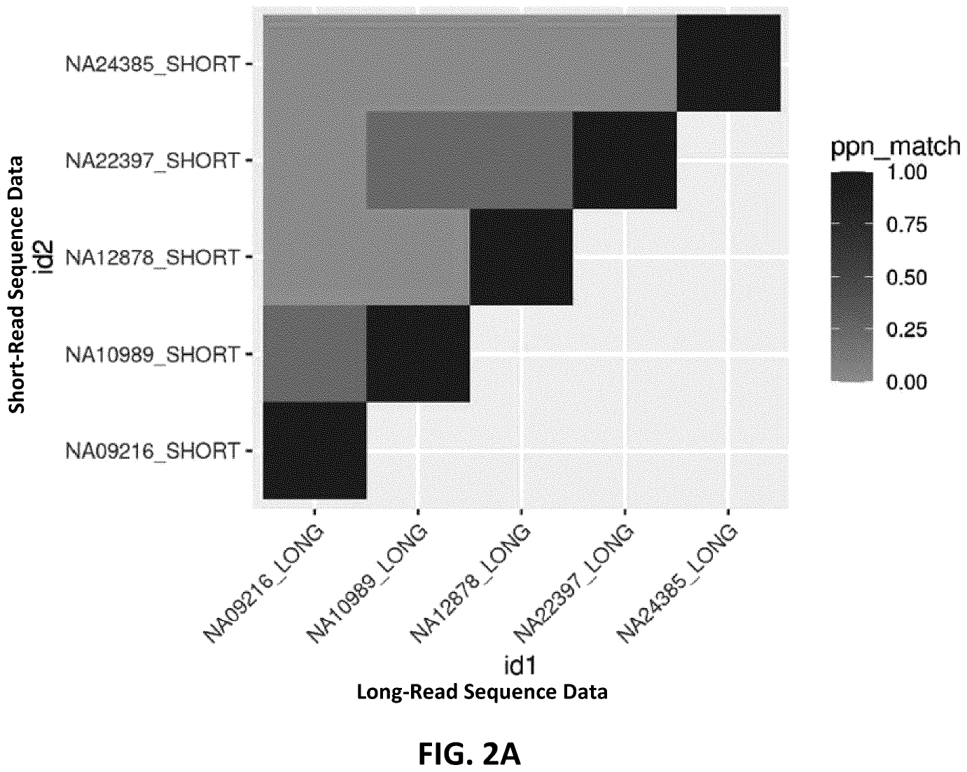

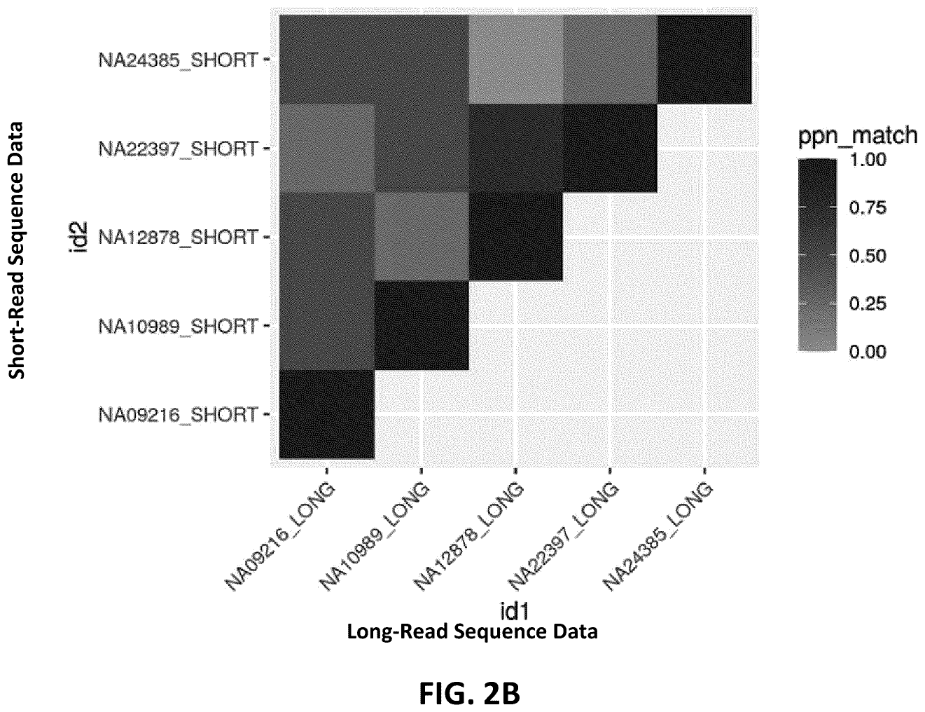

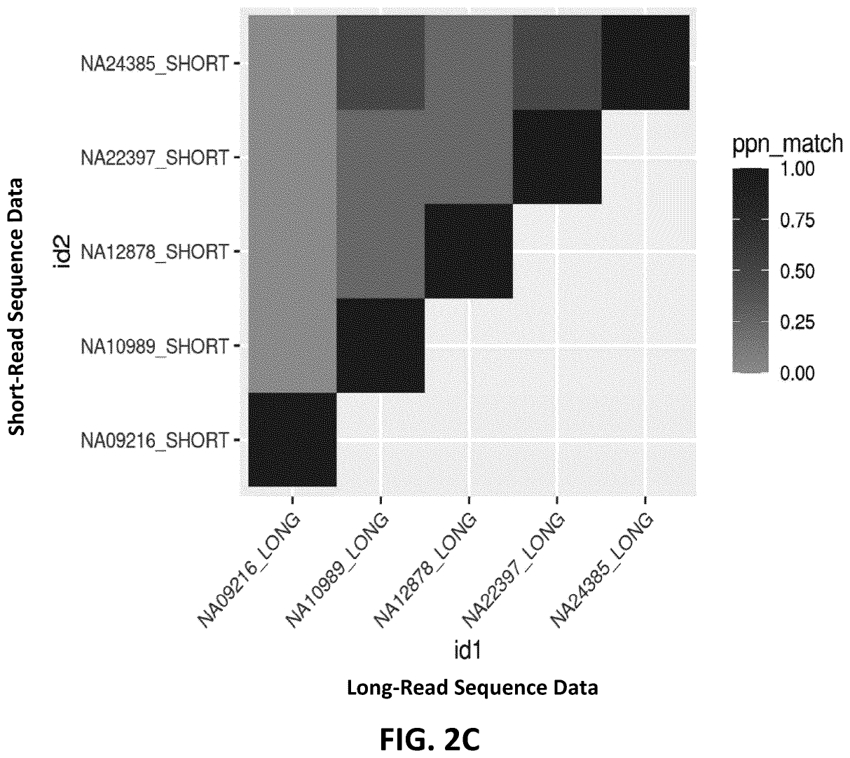

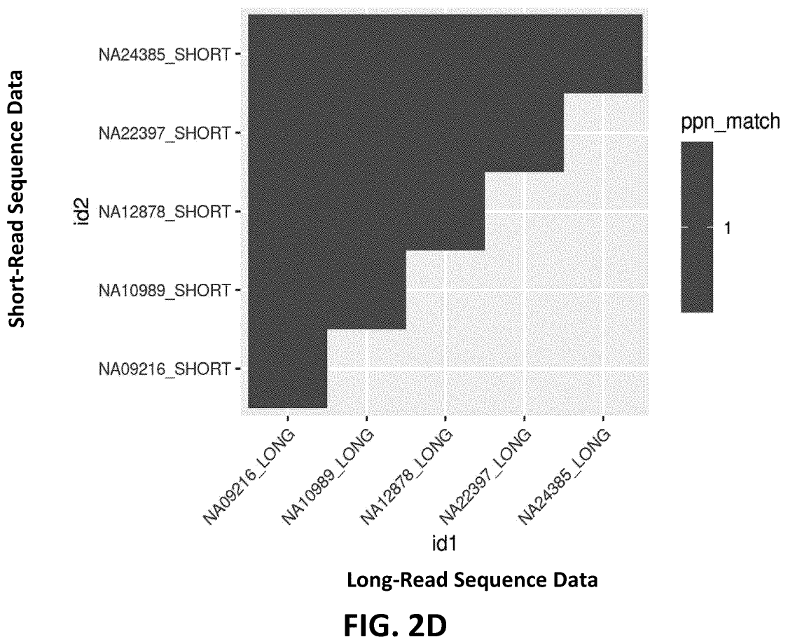

Additionally, FIG. 2A illustrates the proportion of genotypes determined to match-among the 4 SNP loci between the long-read sequence data and the short-read sequence data for each of the 5 samples. As shown in FIG. 2A, each of the samples are determined to have an identical match between their corresponding short-read sequence data and long-read sequence data.

Similarly, FIG. 2B illustrates the proportion of target regions determined to match among the 4 target regions between the long-read sequence data and the short-read sequence data for each of the 5 samples. As shown in FIG. 2B, each of the samples are again determined to have an identical match between their corresponding short-read sequence data and long-read sequence data.

Table 3 summarizes the results of the above using a single SNP method as illustrated in Table 1 and FIG. 2A and the multiple SNP method as illustrated in Table 2 and FIG. 2B. As shown in Table 3, both methods had a sensitivity and specificity of 100%. For sensitivity, each of the five samples had the correct positive comparison between the corresponding short-read sequence and long-read sequence. For specificity, each of the five samples had the correct negative comparison between the respective short-read sequence or long-read sequence and other sample short-read sequences or long-read sequences.

TABLE 3

| | Sensitivity | Specificity |
|---|---|---|
| | Dataset: 5 Coriell samples | Dataset: 5 Coriell samples |
| | (15 comparisons; | (15 comparisons; |
| Method | 5 positive comparisons) | 10 negative comparisons) |
| Single SNP | 100% (5/5) | 100% (10/10) |
| Multiple SNP | 100% (5/5) | 100% (10/10) |

### Example 2

As another example of the accuracy and reliability of the above-described methods for matching samples, this method was again applied to five samples, each sequenced using short-read sequencing and long-read sequencing. In particular, five Coriell cell lines were sequenced using Illumina sequencing (e.g., short-read sequencing) and nanopore sequencing (e.g., long-read sequencing) to generate short-read sequence data and long-read sequence data, respectively.

Similarly, as described in Example 1, Illumina sequencing was performed for each cell line to generate the short-read sequence data for each sample and four target regions were selected based on the short-read sequence data. Here, the SNPs within the four target regions were selected to maximize distance between SNP loci. Prior to long-read sequencing, the four genomic regions corresponding to the four target regions of each sample were enriched and subsequently sequenced using nanopore sequencing to capture the long-read sequence data for each sample.

FIG. 2C illustrates the proportion of target regions determined to match among the 4 target regions between the long-read sequence data and the short-read sequence data for each of the 5 samples. As shown in FIG. 2C, each of the samples are determined to have an identical match between their corresponding short-read sequence data and long-read sequence data.

FIG. 2D illustrates the proportion of target regions that match among the 4 target regions between the long-read sequencing data and the short-read sequencing data if the corresponding SNPs in each target region are randomly selected. As shown in FIG. 2D, the sample matches have no specificity in an instance SNPs are randomly selected, as opposed to selected based on AF as described above in operation 125.

Table 4 summarizes the results of FIGS. 2C-2D using the sample matching method which selects SNPs based on AF as illustrated in FIG. 2C and the sample matching method which selects SNPs randomly as illustrated in FIG. 2D. As shown in Table 4, both methods had a sensitivity of 100%. For sensitivity, each of the five samples had the correct positive comparison between the corresponding short-read sequence and long-read sequence. However, for specificity the AF selected SNPs had a specificity of 100% while the randomly selected SNPs had 0% specificity. As such, each of the five samples which used AF selected SNPs had the correct negative comparison between the respective short-read sequence or long-read sequence and other sample short-read sequences or long-read sequences while randomly selected SNPs did not.

TABLE 4

| | Sensitivity | Specificity |
|---|---|---|
| | Dataset: 5 Coriell samples | Dataset: 5 Coriell samples |
| | (15 comparisons; | (15 comparisons; |
| Method | 5 positive comparisons) | 10 negative comparisons) |
| AF selected | 100% (5/5) | 100% (10/10) |
| SNPs | | |
| Randomly | 100% (5/5) | 0% (0/10) |
| selected SNPs | | |

### Example 3

In general, example embodiments may be deployed in a number of scenarios and/or environments.

For example, one example scenario the above-described sample matching method may be applied in is in verification of sample identities during clinical testing. WGS from short-read data may be commonly available for individuals but may not be sufficient for the purposes of clinical testing. Given a) a set of uniquely identifying SNPs from WGS and b) a DNA sample of this individual, nanopore sequencing may be performed to both identify clinically relevant mutations in this individual as well as confirm that no sample swapping has occurred.

Another example scenario is for use in determination of sample identity in lab research. This sample matching method can be applied to verify the identity of cell lines and patient-derived xenografts in the scenario where multiple sequence datasets are available from each population.

Another example scenario is for sample identification to identify individuals in forensics or crime settings. With this method, DNA can be derived from any source (e.g., blood, hair) to accurately identify the source individuals. Nanopore sequencing with adaptive sampling can be used to target sites necessary to confirm identity in a cost-efficient manner.

As yet another example scenario, the sample matching method may be use with a genome has been sequenced using multiple sequencing technologies and now needs to be matched to combine the findings and benefits from each of the technologies.

### Example 4

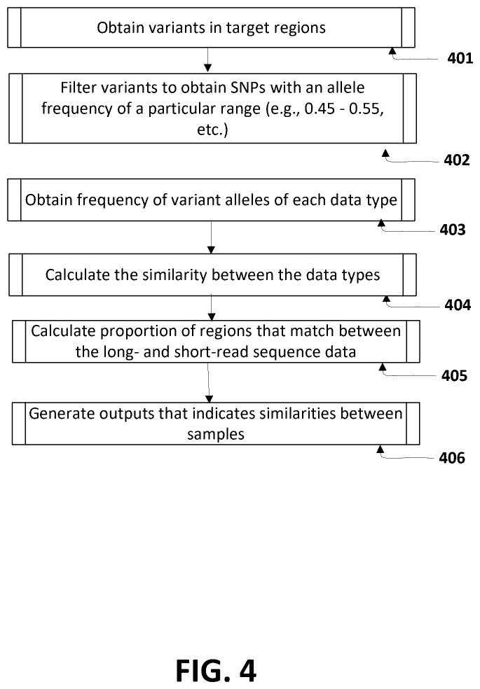

In some embodiments, adaptive sampling is used to perform sample matching. FIG. 4 is a data flow diagram of some embodiments of a process for performing sample matching. The process is performed by processing logic comprising hardware (e.g., processor(s), circuitry, dedicated logic, etc.), software (e.g., software running on a chip, software run on a general-purpose computer system or a dedicated machine, etc.), firmware, or a combination of the three.

Referring to FIG. 4, the process begins by processing logic obtaining variants in target regions (processing block 401) and filters the variants to obtain SNPs with an allele frequency of a particular range (processing block 402). In some embodiments, the variants are obtained from a database, such as for example, the gnomAD. In such a case, processing logic reads the database and pulls files with specific allele frequencies. In some embodiments, the allele frequency range is from 0.45-0.55. Note that other allele frequency ranges can be used.

After filing the variants to obtain SNPs with the desired allele frequency, processing logic obtains frequency of variant alleles of each data type (processing block 403) and calculates the similarity between the data types (processing block 404). In some embodiments, processing logic accesses these variants from bam files and obtains them for both the long-read and short-read sequence data.

Based on the genome type that has been inferred, processing logic calculates the proportion of regions that match between the long- and short-read sequence data (processing block 405). In some embodiments, processing logic determines that variants that have a predetermined proportion of regions that match. In some embodiments, processing logic uses a target region SNP-match threshold of 0.85 (e.g., 85%), such that if 85 of 100 SNPs of a target region are determined to be SNP-matches, processing logic determines that a target region-match exists. In some other embodiments, other target region-match thresholds can be used. For example, if the proportion target regions that match (proportion of sites with matching genome types) is for 30× coverage, then a higher percentage match (e.g., 97-99%) can be used. As part of this operation, processing logic stores information identifying those variants that meet the target region SNP-matching threshold in a buffer.

Thereafter, processing logic generates outputs that indicate similarities between samples (processing block 406). That is, processing logic obtains those variants that meet the SNP-match threshold (e.g., matching greater than 85%, etc.) and generates outputs that show the matching between short- and long-read sequence data. In some embodiments, the outputs include one or more heatmaps, such as, for example, those shown in FIG. 2A-2D.

In some embodiments, the sample matching is performed in parallel with performing the long-read sequencing. For example, if performing long-read sequencing for a particular purpose, the sample matching can be performed in parallel.

Example source code for sample matching methodologies is provided below. The source code below uses adaptive sampling set forth in the flow chart of FIG. 4 described above. Note that the techniques disclosed herein can be performed with other matching methodologies, such as, for example but not limited to, multiplexed sampling.

### CODE
## Get gnomAD variants in target regions
#!/bin/bash
tabix
/references/gnomad/genomes/gnomad.genomes.r2.1.1.sites.8.vcf.bgz
8:1000000-2000000 > chr8.txt
tabix
/references/gnomad/genomes/gnomad.genomes.r2.1.1.sites.9.vcf.bgz
9:50000000-51000000 > chr9.txt
## Filter gnomAD variants to get SNPs with allele frequency 0.45 -
0.55
#!/usr/bin/env python
import sys
import pandas as pd
infile=sys.argv[1]
df=pd.read_table(infile, sep=“\t”, header=None,
names=[‘chr’,‘pos’,‘rs_id’,‘ref’,‘alt’,‘score’,‘filt’,‘info’])
df = df.loc[df[‘filt’]==‘PASS’]
df = df[(df.ref.str.len( )==1) & (df.alt.str.len( )==1)]
def get_af(i):
i_list = i.split(“;”)
af_field = [i for i in i_list if i.startswith(“AF=”)][0]
af = af_field.replace(“AF=”,“”)
return af
df[‘af’] = df[‘info’].apply(lambda x: get_af(x))
df[[‘af’]] = df[[‘af’]]astype(float)
df = df.loc[(df[‘af’]>0.45) & (df[‘af’]<0.55)]
df=df[‘chr’,‘pos’,‘rs_id’,‘ref’,‘alt’,‘score’,‘filt’,‘af’]]
df.to_csv(infile[0:−4] + “.af_filt.txt”, sep=“\t”, index=False)
## Get frequency of variant alleles in bam files of each data type
#!/usr/bin/env python
import sys
import pysam
import pandas as pd
infile_files=“file list.txt”
infile_vars=sys.argv[1]
sample = infile_vars.split(“.”)[0]
df_files = pd.read_table(infile_files, sep=“\t”)
df_files[‘label’] = df_files.apply(lambda row: row[‘id’] + “_” +
row[‘read_type’], axis=1)
df_vars = pd.read_table(infile_vars, sep=“\t”)
out_list = [ ]
for index, row in df_files.iterrows( ):
bam_infile = row[‘file_name’]
id = row[‘label’]
for i, r in df_vars.iterrows( ):
var_chr = str(r[‘chr’])
if row[‘label’] in [‘NA12878_SHORT’,‘NA24385_SHORT’]:
var_chr_mod = “chr” + str(r[‘chr’])
else:
var_chr_mod = str(r[‘chr’])
var_pos = int(r[‘pos’])
var_ref = str(r[‘ref’])
var_alt = str(r[‘alt’])
base_list = [ ]
samfile = pysam.AlignmentFile(bam_infile, “rb” )
for pileupcolumn in samfile.pileup(var_chr_mod, var_pos−1,
var_pos):
if pileupcolumn.pos==var_pos−1:
print(“\ncoverage at base %s = %s” %
(pileupcolumn.pos, pileupcolumn.n))
for pileupread in pileupcolumn.pileups:
if not pileupread.is_del and not
pileupread.is_refskip:
# query position is None if is_del or
is_refskip is set.
print(‘\tbase in read %s = %s’ %
(pileupread.alignment.query_name,
pileupread.alignment.query_sequence[pileupread.query_position]))
base_list.append(pileupread.alignment.query_sequence[pileupread.query
_position])
samfile.close( )
num_reads = len(base_list)
num_ref = base_list.count(var_ref)
num_alt = base_list.count(var_alt)
if num_reads>0:
freq_ref = num_ref/float(num_reads)
freq_alt = num_alt/float(num_reads)
else:
freq_ref = ‘NA’
freq_alt = ‘NA’
out_list.append([id,var_chr,var_pos,var_ref,var_alt,num_reads,num_ref
,num_alt,freq_ref,freq_alt])
df_out = pd.DataFrame(out_list, columns =
[‘id’,‘var_chr’,‘var_pos’,‘var_ref’,‘var_alt’,‘num_reads’,‘num_ref’,‘
num_alt’,‘freq_ref’,‘freq_alt’])
def get_geno(r):
freq_ref = r[‘freq_ref’]
freq_alt = r[‘freq_alt’]
num_reads = r[‘num_reads’]
if num_reads>=10:
if float(freq_ref) >=0.9:
geno=‘hom_ref’
elif float(freq_alt) >=0.9:
geno=‘hom_alt’
else:
geno=‘het’
else:
geno=‘NA’
return geno
df_out[‘geno’] = df_out.apply(lambda row: get_geno(row), axis=1)
df_out[‘var’] = df_out.apply(lambda row: str(row[‘var_chr’]) + “_” +
str(row[‘var_pos’]) + “_” + str(row[‘var_ref]) + “_” +
str(row[‘var_alt’]), axis=1)
df_out.to_csv(sample + “.var_info_all.txt”, sep=“\t”, index=False)
df_out = df_out[[‘id’,‘var’,‘geno’]]
df_out = df_out.set_index([‘id’,
‘var’])[‘geno’].unstack( ).reset_index( )
df_out.columns=df_out.columns.tolist( )
df_out.to_csv(sample + “.var_info.txt”, sep=“\t”, index=False)
## Calculate similarity between data types
#!/usr/bin/env python
import sys
import pandas as pd
from itertools import combinations
infile=sys.argv[1]
sample=infile.split(“.”)[0]
df = pd.read_table(infile, sep=“\t”)
df_list = df.values.tolist( )
list_combinations = list( )
for n in range(len(df_list) + 1):
list_combinations += list(combinations(df_list, n))
list_combinations_2 = [1 for 1 in list_combinations if
len(1) == 2]
print(list_combinations_2)
out_list=[ ]
for i in list_combinations_2:
list1 = i[0]
list2 = i[1]
id1 = list1[0]
id2 = list2[0]
geno1 = list1[1:101]
geno2 = list2[1:101]
num_vars = 0
num_match = 0
for x,y in zip(geno1,geno2):
if (str(x)!=‘nan’ and str(y)!=‘nan’):
num_vars=num_vars+1
if x==y:
num_match=num_match+1
ppn_match = num_match/float(num_vars)
out_list.append([id1,id2,num_vars,num_match,ppn_match])
df_out = pd.DataFrame(out_list, columns =
[‘id1’,‘id2’,‘num_vars’,‘num_match’,‘ppn_match’])
df_out = df_out[df_out[‘id1’].str.contains(‘LONG’)]
df_out = df_out[df_out[‘id2’].str.contains(‘SHORT’)]
df_out.to_csv(sample + ‘.heatmap.txt’, sep=“\t”, index=False)
## Calculate proportion of regions that match
#!/usr/bin/env python
import sys
import pandas as pd
import glob
from functools import reduce
files = glob.glob(“*.heatmap.txt”)
sample_list = [ ]
df_list=[ ]
for infile in files:
sample = infile.split(“.”)[0]
df = pd.read_table(infile, sep=“\t”)
df[sample] = df[‘ppn_match’].apply(lambda x: 1 if x>0.85 else
0)
sample_list.append(sample)
df = df[[‘id1’,‘id2’,sample]]
df_list.append(df)
print(df)
df_out = reduce(lambda left,right:
pd.merge(left,right,on=[‘id1’,‘id2’],
how=‘outer’), df_list)
df_out[‘num_match’]= df_out[sample_list].sum(axis=1)
df_out[‘ppn_match’] = df_out[‘num_match’].apply(lambda x:
float(x)/float(4))
df out.to_csv(‘heatmap_input.txt’, sep=“\t”, index=False)
## Plot heatmap
#!/usr/bin/env Rscript
library(ggplot2)
infile <−‘heatmap_input.txt’
df <−read.table(infile, sep=“\t”, header=T)
ggplot(df, aes(id1, id2, fill= ppn_match)) +
geom_tile( ) +
scale_fill_continuous(high = “#132B43”, low = “#56B1F7”) +
theme(axis.text.x = element_text(angle = 45, vjust = 1, hjust=1))
ggsave(“snp_id_heatmap.png”, height=4, width=5.5)

Note that for the techniques above, the base sequences can be determined one or more ways. For example, in some embodiments, Oxford Nanopore sequencing technology (ONT) uses nanopore technology to determine the base sequences. However, the techniques disclosed herein are not limited to ONT, and other technologies can be used to determine the base sequences. Also in some other embodiments, long-read whole genome sequencing can be used with any long-read platform.

There are a number of example embodiments described herein.

Example 1 is a method for sequence data sample matching that includes receiving short-read sequence data, wherein the short-read sequence data is generated from a first sample using a short-read sequencing technique; identifying a plurality of short-read single nucleotide polymorphisms (SNPs) from the short-read sequence data; selecting one or more SNPs from the plurality of short-read SNPs; receiving long-read sequence data, wherein the long-read sequence data is generated from a second sample using a long-read sequencing technique, wherein the long-read sequence data comprises sequence data for at least the one or more SNPs, and determining whether the first sample and the second sample match based on the short-read sequence data and the long-read sequence data.

Example 2 is the method of example 1 that may optionally include annotating each short-read SNP of the plurality of short-read SNPs with a corresponding allele frequency and selecting the one or more SNPs based on allele frequency of the plurality of short-read SNPs.

Example 3 is the method of example 2 that may optionally include determining one or more population allele frequency specific SNPs based on population SNP data, wherein selecting the one or more SNPs is further based on the one or more population allele frequency specific SNPs.

Example 4 is the method of example 2 that may optionally include determining one or more subpopulation allele frequency specific SNPs based on subpopulation SNP data, wherein the subpopulation SNP data corresponds to a particular subpopulation of the first sample, wherein selecting the one or more SNPs is further based on the one or more subpopulation allele frequency specific SNPs.

Example 5 is the method of example 2 that may optionally include determining one or more rare SNPs based on rare SNP data, wherein the rare SNP data corresponds to an allele frequency that occurs in less than a threshold percentage of a population, wherein selecting the one or more SNPs is further based on the one or more rare SNPs.

Example 6 is the method of example 5 that may optionally include that the threshold percentage is less than 0.05 percent of the population.

Example 7 is the method of example 1 that may optionally include that each short-read SNP is associated with an SNP locus indicative of a genomic location of the short-read SNP on the first sample, wherein the method further includes: identifying a corresponding SNP described by the long-read sequence data for each SNP described by the short-read sequence data based on the associated SNP locus, comparing each SNP described by the short-read sequence data to its corresponding SNP described by the long-read sequence data; and determining a number of SNP-matches, wherein a SNP-match is determined in an instance in which a SNP at a particular SNP locus described by the short-read sequence matches a SNP at a corresponding SNP locus described by the long-read sequence data.

Example 8 is the method of example 7 that may optionally include determining that the first sample and the second sample match in an instance in which the number of SNP-matches satisfies one or more SNP-match thresholds.

Example 9 is the method of example 1 that may optionally include determining one or more target regions for long-read sequencing based on the one or more selected SNPs, wherein the long-read sequence data comprises sequence data for at least the one or more target regions and wherein each target region includes one or more of the one or more SNPs.

Example 10 is the method of example 9 that may optionally include that each short-read SNP is associated with an SNP locus indicative of a genomic location of the short-read SNP on the first sample, wherein the method further includes: determining a number of target region-matches, wherein determining a target region match for a target region of the one or more target regions includes: for each SNP in the target region, identifying a corresponding SNP described by the long-read sequence data for each SNP described by the short-read sequence data based on the associated SNP locus; comparing each SNP described by the short-read sequence data to its corresponding SNP described by the long-read sequence data; determining a number of target region SNP-matches for a target region, wherein a target region SNP-match is determined in an instance in which a SNP at a particular SNP locus within the target region described by the short-read sequence matches a SNP at a corresponding SNP locus described by the long-read sequence data; and determining a target region-match in an instance in which the number of target region SNP-matches satisfies one or more target region SNP-match thresholds.

Example 11 is the method of example 10 that may optionally include determining that the first sample and the second sample match in an instance in which the number of target region-matches satisfies one or more target region-match thresholds.

Example 12 is the method of example 1 that may optionally include that the one or more SNPs are selected based on a distance between respective SNP locus sites.

Example 13 is the method of example 12 that may optionally include that the one or more SNPs are selected such that the distance between SNP loci is maximized.

Example 14 is the method of example 1 that may optionally include providing a sample determination response, wherein the sample determination response is indicative of whether the first sample and second sample match.

Example 15 is a method for determining sample matches that includes: performing a short-read sequencing technique on a first sample to generate short-read sequence data; identifying a plurality of short-read single nucleotide polymorphisms (SNPs) from the short-read sequence data; annotating each short-read SNP of the plurality of short-read SNPs with a corresponding allele frequency: selecting one or more SNPs from the plurality of short-read SNPs; and determining one or more target regions for long-read sequencing based on the one or more SNPs.

Example 16 is the method of example 15 that may optionally include that performing a short-read sequencing technique on the first sample comprises: (i) fragmenting the first sample into two or more fragments, (ii) amplifying the two or more fragments, and (iii) generating the short-read sequence data based on sequencing the two or more amplified fragments.

Example 17 is the method of example 15 that may optionally include selecting the one or more SNPs based on allele frequency of the plurality of short-read SNPs.

Example 18 is the method of example 17 that may optionally include determining one or more population allele frequency specific SNPs based on population SNP data, wherein selecting the one or more SNPs is further based on the one or more population allele frequency specific SNPs.

Example 19 is the method of example 17 that may optionally include determining one or more subpopulation allele frequency specific SNPs based on subpopulation SNP data, wherein the subpopulation SNP data corresponds to a particular subpopulation of the first sample, wherein selecting the one or more SNPs is further based on the one or more subpopulation allele frequency specific SNPs.

Example 20 is the method of example 17 that may optionally include determining one or more rare SNPs based on rare SNP data, wherein the rare SNP data corresponds to an allele frequency that occurs in less than a threshold percentage of a population, wherein selecting the one or more SNPs is further based on the one or more rare SNPs.

Example 21 is the method of example 20 that may optionally include that the threshold percentage is less than 0.05 percent of the population.

Example 22 is the method of example 15 that may optionally include performing a long-read sequencing technique on a second sample to obtain long-read sequence data, wherein the long-read sequencing includes sequencing of the one or more target regions.

Example 23 is the method of example 22 that may optionally include that the long-read sequencing technique is nanopore sequencing.

Example 24 is the method of example 22 that may optionally include that each short-read SNP is associated with an SNP locus indicative of a genomic location of the short-read SNP on the first sample, wherein the method further includes: identifying a corresponding SNP described by the long-read sequence data for each SNP described by the short-read sequence data based on the associated SNP locus, comparing each SNP described by the short-read sequence data to its corresponding SNP described by the long-read sequence data, and determining a number of SNP-matches, wherein a SNP-match is determined in an instance in which a SNP at a particular SNP locus described by the short-read sequence matches a SNP at a corresponding SNP locus described by the long-read sequence data.

Example 25 is the method of example 24 that may optionally include determining that the first sample and the second sample match in an instance in which the number of SNP-matches satisfies one or more SNP-match thresholds.

Example 26 is the method of example 22 that may optionally include determining one or more target regions for long-read sequencing based on the one or more selected SNPs, wherein the long-read sequence data comprises sequence data for at least the one or more target regions and wherein each target region includes one or more of the one or more SNPs.

Example 27 is the method of example 26 that may optionally include that each short-read SNP is associated with an SNP locus indicative of a genomic location of the short-read SNP on the first sample, wherein the method further includes: determining a number of target region-matches, wherein determining a target region match for a target region of the one or more target regions comprises: for each SNP in the target region, identifying a corresponding SNP described by the long-read sequence data for each SNP described by the short-read sequence data based on the associated SNP locus, comparing each SNP described by the short-read sequence data to its corresponding SNP described by the long-read sequence data: determining a number of target region SNP-matches for a target region, wherein a target region SNP-match is determined in an instance in which a SNP at a particular SNP locus within the target region described by the short-read sequence matches a SNP at a corresponding SNP locus described by the long-read sequence data; and determining a target region-match in an instance in which the number of target region SNP-matches satisfies one or more target region SNP-match thresholds.

Example 28 is the method of example 27 that may optionally include determining that the first sample and the second sample match in an instance in which the number of target region-matches satisfies one or more target region-match thresholds.

Example 29 is the method of example 15 that may optionally include that selecting the one or more SNPs is based on a distance between respective SNP locus sites.

Example 30 is the method of example 29 that may optionally include that the one or more SNPs are selected such that the distance between SNP loci is maximized.

Example 31 is the method of example 15 that may optionally include providing a sample determination response, wherein the sample determination response is indicative of whether the first sample and second sample match.

Example 32 is an apparatus for determining sample matches that includes a processor and a memory storing software instructions that, when executed by the processor, cause the apparatus to perform the steps recited in any of examples 1 to 31.

Example 33 is a computer program product for determining sample matches that includes at least one non-transitory computer-readable storage medium storing software instructions that, when executed, cause an apparatus to perform the steps recited in any of examples 1 to 31.

### CONCLUSION

Many modifications and other embodiments of the inventions set forth herein will come to mind to one skilled in the art to which these inventions pertain having the benefit of the teachings presented in the foregoing descriptions and the associated drawings. Therefore, it is to be understood that the inventions are not to be limited to the specific embodiments disclosed and that modifications and other embodiments are intended to be included within the scope of the appended claims. Moreover, although the foregoing descriptions and the associated drawings describe example embodiments in the context of certain example combinations of elements and/or functions, it should be appreciated that different combinations of elements and/or functions may be provided by alternative embodiments without departing from the scope of the appended claims. In this regard, for example, different combinations of elements and/or functions than those explicitly described above are also contemplated as may be set forth in some of the appended claims. Although specific terms are employed herein, they are used in a generic and descriptive sense only and not for purposes of limitation.

All patents and publications mentioned in the specification are indicative of the levels of those of ordinary skill in the art to which the invention pertains. All patents and publications are herein incorporated by reference to the same extent as if each individual publication was specifically and individually indicated to be incorporated by reference.

What is claimed is:

1. A method for sequence data sample matching, the method comprising:
receiving short-read sequence data, wherein the short-read sequence data is generated from a first sample using a short-read sequencing technique;
identifying a plurality of short-read single nucleotide polymorphisms (SNPs) from the short-read sequence data;
selecting one or more SNPs from the plurality of short-read SNPs;
receiving long-read sequence data, wherein the long-read sequence data is generated from a second sample using a long-read sequencing technique, wherein the long-read sequence data comprises sequence data for at least the one or more SNPs; and
determining whether the first sample and the second sample match based on the short-read sequence data and the long-read sequence data.

2. The method of claim 1, further comprising:
annotating each short-read SNP of the plurality of short-read SNPs with a corresponding allele frequency; and
selecting the one or more SNPs based on allele frequency of the plurality of short-read SNPs.

3. The method of claim 2, further comprising:
determining one or more population allele frequency specific SNPs based on population SNP data,
wherein selecting the one or more SNPs is further based on the one or more population allele frequency specific SNPs.

4. The method of claim 2, further comprising:
determining one or more subpopulation allele frequency specific SNPs based on subpopulation SNP data, wherein the subpopulation SNP data corresponds to a particular subpopulation of the first sample, wherein selecting the one or more SNPs is further based on the one or more subpopulation allele frequency specific SNPs.

5. The method of claim 2, further comprising:
determining one or more rare SNPs based on rare SNP data, wherein the rare SNP data corresponds to an allele frequency that occurs in less than a threshold percentage of a population, wherein selecting the one or more SNPs is further based on the one or more rare SNPs.

6. The method of claim 5, wherein the threshold percentage is less than 0.05 percent of the population.

7. The method of claim 1, wherein each short-read SNP is associated with an SNP locus indicative of a genomic location of the short-read SNP on the first sample, wherein the method further comprises:
identifying a corresponding SNP described by the long-read sequence data for each SNP described by the short-read sequence data based on the associated SNP locus;
comparing each SNP described by the short-read sequence data to its corresponding SNP described by the long-read sequence data; and
determining a number of SNP-matches, wherein a SNP-match is determined in an instance in which a SNP at a particular SNP locus described by the short-read sequence matches a SNP at a corresponding SNP locus described by the long-read sequence data.

8. The method of claim 7, further comprising:
determining that the first sample and the second sample match in an instance in which the number of SNP-matches satisfies one or more SNP-match thresholds.

9. The method of claim 1, further comprising determining one or more target regions for long-read sequencing based on the one or more selected SNPs, wherein the long-read sequence data comprises sequence data for at least the one or more target regions and wherein each target region includes one or more of the one or more SNPs.

10. The method of claim 9, wherein each short-read SNP is associated with an SNP locus indicative of a genomic location of the short-read SNP on the first sample, wherein the method further comprises:
determining a number of target region-matches, wherein determining a target region match for a target region of the one or more target regions comprises:
for each SNP in the target region, identifying a corresponding SNP described by the long-read sequence data for each SNP described by the short-read sequence data based on the associated SNP locus;
comparing each SNP described by the short-read sequence data to its corresponding SNP described by the long-read sequence data;
determining a number of target region SNP-matches for a target region, wherein a target region SNP-match is determined in an instance in which a SNP at a particular SNP locus within the target region described by the short-read sequence matches a SNP at a corresponding SNP locus described by the long-read sequence data; and
determining a target region-match in an instance in which the number of target region SNP-matches satisfies one or more target region SNP-match thresholds.

11. The method of claim 10, further comprising:
determining that the first sample and the second sample match in an instance in which the number of target region-matches satisfies one or more target region-match thresholds.

12. The method of claim 1, wherein the one or more SNPs are selected based on a distance between respective SNP locus sites.

13. The method of claim 12, wherein the one or more SNPs are selected such that the distance between SNP loci is maximized.

14. The method of claim 1, further comprising:
providing a sample determination response, wherein the sample determination response is indicative of whether the first sample and second sample match.

15. The method of claim 1, further comprising performing the short-read sequencing technique on the first sample to generate the short-read sequence data.

16. The method of claim 1, wherein performing a short-read sequencing technique on the first sample comprises: (i) fragmenting the first sample into two or more fragments, (ii) amplifying the two or more fragments, and (iii) generating the short-read sequence data based on sequencing the two or more amplified fragments.

17.-21. (canceled)

22. The method of claim 9, further comprising performing the long-read sequencing technique on the second sample to obtain the long-read sequence data, wherein the long-read sequencing includes sequencing of the one or more target regions.

23. The method of claim 1, wherein the long-read sequencing technique is nanopore sequencing.

24.-31. (canceled)

32. An apparatus for determining sample matches, the apparatus comprising a processor and a memory storing software instructions that, when executed by the processor, cause the apparatus to perform a method, the method comprising:
receiving short-read sequence data, wherein the short-read sequence data is generated from a first sample using a short-read sequencing technique;
identifying a plurality of short-read single nucleotide polymorphisms (SNPs) from the short-read sequence data;
selecting one or more SNPs from the plurality of short-read SNPs;
receiving long-read sequence data, wherein the long-read sequence data is generated from a second sample using a long-read sequencing technique, wherein the long-read sequence data comprises sequence data for at least the one or more SNPs; and
determining whether the first sample and the second sample match based on the short-read sequence data and the long-read sequence data.

33. A computer program product for determining sample matches, the computer program product comprising at least one non-transitory computer-readable storage medium storing software instructions that, when executed, cause an apparatus to perform a method, the method comprising:
receiving short-read sequence data, wherein the short-read sequence data is generated from a first sample using a short-read sequencing technique;
identifying a plurality of short-read single nucleotide polymorphisms (SNPs) from the short-read sequence data;
selecting one or more SNPs from the plurality of short-read SNPs;
receiving long-read sequence data, wherein the long-read sequence data is generated from a second sample using a long-read sequencing technique, wherein the long-read sequence data comprises sequence data for at least the one or more SNPs; and
determining whether the first sample and the second sample match based on the short-read sequence data and the long-read sequence data.
